(19) United States
(12) Patent Application Publication
(10) Pub. No.: US 2020/0324086 A1
(43) Pub. Date: Oct. 15, 2020

(54) INTRODUCER NEEDLE AND RELATED SYSTEMS AND METHODS

(71) Applicant: Becton, Dickinson and Company, Franklin lakes, NJ (US)

(72) Inventor: S. Ray Isaacson, Layton, UT (US)

(21) Appl. No.: 16/846,956

(22) Filed: Apr. 13, 2020

Related U.S. Application Data
(60) Provisional application No. 62/834,233, filed on Apr. 15, 2019.

(51) Int. Cl. A61M 25/06

(52) U.S. Cl. CPC A61M 25/065; A61M 25/0693

(57) ABSTRACT

A distal end of a needle may include a distal tip. The distal end may also include a first beveled surface, a second beveled surface, and a third beveled surface, which may each extend proximally from the distal tip. The first beveled surface may converge with the second beveled surface to form a first cutting edge, the second beveled surface may converge with the third beveled surface to form a second cutting edge, and the third beveled surface may converge with the first beveled surface to form a third cutting edge. The first cutting edge may be offset from the second cutting edge by about 120 degrees, the second cutting edge may be offset from the third cutting edge by about 120 degrees, and the third cutting edge may be offset from the first cutting edge by about 120 degrees.

### RELATED APPLICATIONS

This application claims the benefit of United States Application No. 62/834,233, filed Apr. 15, 2019, and entitled INTRODUCER NEEDLE AND RELATED SYSTEMS AND METHODS, which is incorporated herein in its entirety.

### BACKGROUND

Catheters are generally used for parenteral nutrition, intravenous fluid replacement, and administering analgesics and antibiotics. Catheters are also used for blood draw. Catheters can be inserted at the bedside using sterile techniques and can remain in place for several weeks.

A common type catheter is an over-the-needle catheter. As its name implies, a catheter that is “over-the-needle” may be mounted over an introducer needle having a sharp distal tip. The sharp distal tip may be used to pierce skin and a vein of a patient. Insertion of the over-the-needle catheter into the vein may follow the piercing of the vein by the introducer needle. The introducer needle typically has the sharp distal tip to pierce skin and the vein of the patient with minimal resistance to minimize the pain to the patient.

The introducer needle is generally placed at a steep inclined angle with respect to a surface of the skin and a longitudinal dimension of the vein to be pierced to allow penetration through the skin and a wall of the vein. The needle and the catheter are generally inserted with a bevel of the introducer needle facing away from the skin of the patient. After the tip of the introducer needle pierces the wall, the angle of the insertion is lowered to be able to slide the introducer needle and the catheter into the vein a distance sufficient to properly position the catheter in the vein. Once placement of the introducer needle within the vein has been confirmed, the user may temporarily occlude flow in the vein and withdraw the introducer needle, leaving the over-the-needle catheter in place for future fluid infusion and/or blood withdrawal.

Inserting and properly positioning the catheter on the first attempt generally requires a level of skill that is not possessed by some users. The accurate placement of the catheter in the vein on the first attempt is a great advantage to reduce the incidence of damage to the vein and/or the surrounding tissue. Proper placement of the catheter and the needle is important to minimize discomfort and pain to the patient as well as reducing damage or injury to the vein. Damage caused to the vein during insertion and placement of the catheter can cause accelerated clotting, thrombosis, and other health problems.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access systems, devices, and methods. More particularly, in some embodiments, the present disclosure relates to needles and related devices, systems, and methods.

In some embodiments, the needle may include an introducer needle. In some embodiments, a needle tip geometry of the needle may provide a reduced risk of transfixing a vein of a patient when the needle is inserted into the vein of the patient. In some embodiments, the needle tip geometry may provide more reliable placement of the catheter within the vein. In some embodiments, the needle may be used to insert a guidewire into the patient for advancing the catheter.

In some embodiments, the needle may include a distal end, a proximal end, and an elongated body extending between the distal end and the proximal end. In some embodiments, the distal end of the needle may include a distal tip, a first beveled surface extending proximally from the distal tip, a second beveled surface extending proximally from the distal tip, and a third beveled surface extending proximally from the distal tip. In some embodiments, the distal tip may include a sharp point for penetrating skin and the vein of the patient.

In some embodiments, the first beveled surface may converge with the second beveled surface to form a first cutting edge. In some embodiments, the second beveled surface may converge with the third beveled surface to form a second cutting edge. In some embodiments, the third beveled surface may converge with the first beveled surface to form a third cutting edge. In some embodiments, the first cutting edge may be offset from the second cutting edge by about 120 degrees. In some embodiments, the second cutting edge may be offset from the third cutting edge by about 120 degrees. In some embodiments, the third cutting edge may be offset from the first cutting edge by about 120 degrees.

In some embodiments, the needle may be solid. In some embodiments, the needle may include one or more flash grooves. In some embodiments, the flash grooves may extend proximally from one or more of the first beveled surface, the second beveled surface, and the third beveled surface.

In some embodiments, the needle may include a lumen extending through the proximal end of the needle. In some embodiments, one or more of the first beveled surface, the second beveled surface, or the third beveled surface may include one or more holes in fluid communication with the lumen of the needle. In some embodiments, the guidewire may be disposed within the lumen of the needle and may be advanced distally through a particular one of the holes beyond the distal tip of the needle.

In some embodiments, a perimeter of the first beveled surface (which may be referred to in the present disclosure as the first perimeter) may include the first cutting edge, the third cutting edge, and a first proximal edge. In some embodiments, the first proximal edge may extend between the first cutting edge and the third cutting edge. In some embodiments, a perimeter of the second beveled surface (which may be referred to in the present disclosure as the second perimeter) may include the first cutting edge, the second cutting edge, and a second proximal edge. In some embodiments, the second proximal edge may extend between the second cutting edge and the third cutting edge. In some embodiments, a perimeter of the third beveled surface (which may be referred to in the present disclosure as the third perimeter) may include the second cutting edge, the third cutting edge, and a third proximal edge. In some embodiments, the third proximal edge may extend between the second cutting edge and the third cutting edge.

In some embodiments, a first portion of the elongated body disposed between the first proximal edge and the second proximal edge may be chamfered. In some embodiments, a second portion of the elongated body between the second proximal edge and the third proximal edge may be chamfered. In some embodiments, a third portion of the elongated body disposed between the second proximal edge and the third proximal edge may be chamfered.

In some embodiments, the distal end of the needle may be symmetric. In some embodiments, the first perimeter, the second perimeter, and the third perimeter may be equal. In these and other embodiments, the distal tip may be aligned with a central axis of the needle. In some embodiments, a length of the first beveled surface from the distal tip to an apex of the first proximal edge may be equal to a length of the second beveled surface from the distal tip to an apex of the second proximal edge, which may be equal to a length of the third beveled surface from the distal tip to an apex of the third proximal edge. In some embodiments, a length of the first beveled surface from a proximal end of the first cutting edge to a proximal end of the third cutting edge may be equal to a length of the second beveled surface from a proximal end of the first cutting edge to a proximal end of a second cutting edge, which may be equal to a length of the third beveled surface from the proximal end of the second cutting edge to the proximal end of the third cutting edge.

In some embodiments, the distal end of the needle may be asymmetric. In some embodiments, one or more of the first perimeter, the second perimeter, and the third perimeter may not be equal to each other. In these and other embodiments, the distal tip may not be aligned with a central axis of the needle. In some embodiments, a length of the first beveled surface from the distal tip to an apex of the first proximal edge may be greater than a length of the second beveled surface from the distal tip to an apex of the second proximal edge and/or a length of the third beveled surface from the distal tip to an apex of the third proximal edge. In some embodiments, a length of the second beveled surface from the distal tip to an apex of the second proximal edge may be greater than a length of the first beveled surface from the distal tip to an apex of the first proximal edge and/or a length of the third beveled surface from the distal tip to an apex of the third proximal edge. In some embodiments, a length of the third beveled surface from the distal tip to an apex of the third proximal edge may be greater than a length of the second beveled surface from the distal tip to an apex of the second proximal edge and/or a length of the first beveled surface from the distal tip to an apex of the first proximal edge.

In some embodiments, the needle may extend through a catheter of a catheter insertion device, although it is contemplated that the needle may be used with non-catheter medical devices and/or for any medical purpose, including as a spinal needle. In some embodiments, the catheter may include a peripheral intravenous catheter (PIVC), a midline catheter, or a peripherally inserted central catheter (PICC).

In some embodiments, the catheter may include a distal end, a proximal end, and a lumen extending through the distal end of the catheter and the proximal end of the catheter. In some embodiments, the flash grooves may each include a distal end proximate the first beveled surface, the second beveled surface, or the third beveled surface. In some embodiments, the flash grooves may each include a proximal end disposed proximal to the distal end of the catheter. In some embodiments, the flash grooves may be in fluid communication with the lumen of the catheter. In some embodiments, blood traveling through the flash grooves and into the lumen of the catheter may be visible to a user.

In some embodiments, one or more of the first beveled surface, the second beveled surface, and the third beveled surface may be generally planar. In some embodiments, the needle may be oriented within the catheter such that the first beveled surface, the second beveled surface, or the third beveled surface faces downwardly or towards an interior wall of the vein opposite a point of entry of the needle into the vein. In these embodiments, a particular cutting edge facing toward a top of the catheter may be aligned with a central axis of the catheter insertion device. In some embodiments, when the needle is inserted into the vein with a particular beveled surface facing downwardly, a risk of transfixing the vein may be reduced.

In some embodiments, the needle tip geometry may be used on a blood draw phlebotomy needle for withdrawing blood through a lumen of the needle into a vacuum tube or other suitable blood collection device. In some embodiments, the needle tip geometry may also be used for other procedures, such as, for example, procedures where it is desired to reduce needle bending caused by a long bevel needle. In some embodiments, the needle may be used for introducing and/or withdrawing fluid from a patient. In some embodiments, the needle tip geometry may be used for aspiration of blood, spinal fluid, or other fluids from a patient.

It is to be understood that both the foregoing general description and the following detailed description are exemplary and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example needle, according to some embodiments;

FIG. 1B is a lower perspective view of the needle of FIG. 1A, according to some embodiments;

FIG. 1C is a side view of the needle of FIG. 1A, according to some embodiments;

FIG. 1D is an opposite side view of the needle of FIG. 1A, according to some embodiments;

FIG. 1E is a perspective view of the needle of FIG. 1A from a distal end of the needle, according to some embodiments;

FIG. 1F is an upper perspective view of the needle of FIG. 1A, illustrating the needle in comparison with another needle, according to some embodiments;

FIG. 1G is an upper perspective view of the needle of FIG. 1A inserted into a vein, illustrating the needle in comparison with the other needle, according to some embodiments;

FIG. 2A is an upper perspective view of the needle of FIG. 1A, illustrating an example flash groove, according to some embodiments;

FIG. 2B is a side view of the needle of FIG. 1A, illustrating multiple flash grooves, according to some embodiments;

FIG. 2C is a perspective view of the needle of FIG. 1A from the distal end of the needle, illustrating multiple flash grooves, according to some embodiments;

FIG. 3A is a top view of the needle of FIG. 1A, illustrating example chamfered portions, according to some embodiments;

FIG. 3B is a perspective view of the needle of FIG. 1A from the distal end of the needle, illustrating multiple chamfered portions, according to some embodiments;

FIG. 4A is an upper perspective view of the needle of FIG. 1A, illustrating an example hole, according to some embodiments;

FIG. 4B is an upper perspective view of the needle of FIG. 1A inserted through an example catheter, according to some embodiments;

FIG. 5 is a top view of the needle of FIG. 1A inserted through the catheter, illustrating an example notch, according to some embodiments;

FIG. 6A is an upper perspective view of the needle of FIG. 1A, illustrating an asymmetric distal end, according to some embodiments;

FIG. 6B is an upper perspective view of the needle of FIG. 1A, illustrating another asymmetric distal end, according to some embodiments;

FIG. 7A is an exploded view of an example catheter assembly, according to some embodiments;

FIG. 7B is a partial cross-sectional view of the catheter assembly, according to some embodiments;

FIG. 7C is an upper perspective view of the catheter assembly, illustrating example blood flashback, according to some embodiments;

FIG. 7D is an upper perspective view of another catheter assembly, according to some embodiments;

FIG. 7E is a cross-sectional view of the other catheter assembly, according to some embodiments;

FIG. 8A is a top view of the needle, illustrating two beveled surfaces, according to some embodiments;

FIG. 8B is a bottom view of the needle, illustrating two beveled surfaces, according to some embodiments;

FIG. 8C is a side view of the needle, illustrating two beveled surfaces, according to some embodiments;

FIG. 8D is a perspective view from a distal end of the needle, illustrating two beveled surfaces, according to some embodiments,

FIG. 9A is a side view of the needle, illustrating four beveled surfaces, according to some embodiments; and

FIG. 9B is a perspective view from a distal end of the needle, illustrating four beveled surfaces, according to some embodiments.

### DESCRIPTION OF EMBODIMENTS

As used in the present disclosure, the term “distal” refers to a portion of a needle or a catheter assembly or component thereof that is farther from a user, and the term “proximal” refers to a portion of a needle or a catheter assembly or component thereof that is closer to the user. As used in the present disclosure, the term “user” may refer to a clinician, doctor, nurse, or any other care provider and may include support personnel.

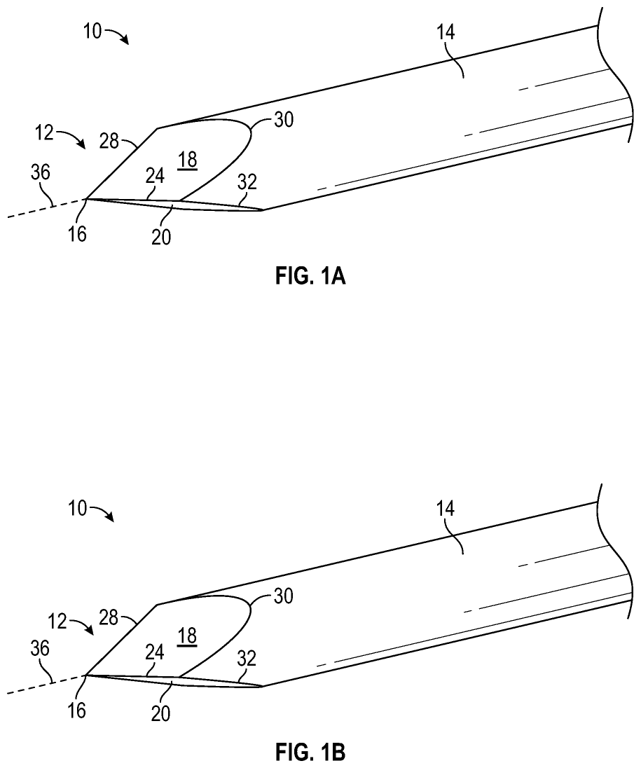

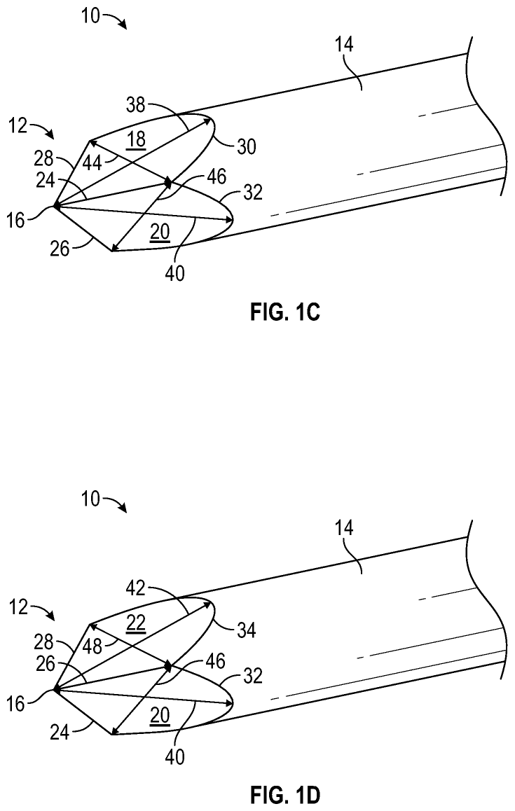

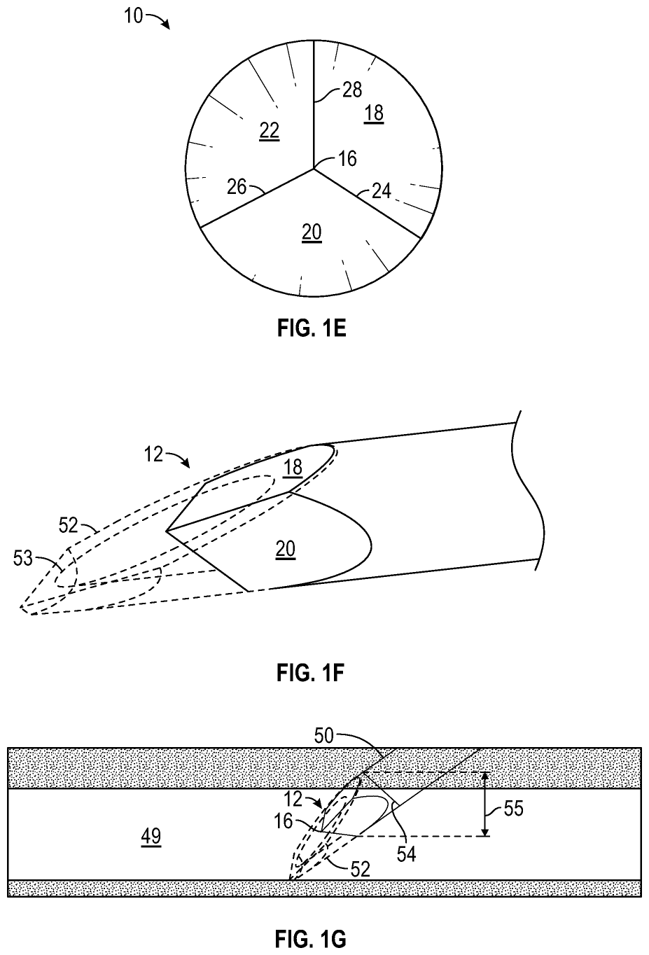

Referring now to FIGS. 1A-1E, in some embodiments, a needle 10 may include an introducer needle. In some embodiments, the needle 10 may include a distal end 12, a proximal end, and an elongated body 14 extending between the distal end 12 and the proximal end. In some embodiments, the elongated body 14 may be generally cylindrical, which may prevent catching, or another suitable shape. In some embodiments, the distal end 12 may be generally symmetrical, as illustrated, for example, in FIGS. 1A-1E.

In some embodiments, the distal end 12 of the needle 10 may include a distal tip 16, a first beveled surface 18 extending proximally from the distal tip 16, a second beveled surface 20 extending proximally from the distal tip 16, and a third beveled surface 22 extending proximally from the distal tip 16. In some embodiments, the distal tip 16 may include a sharp point for penetrating skin and the vein of the patient. In some embodiments, the distal tip 16 may be disposed radially inward from an outer surface of the elongated body 14.

In some embodiments, the first beveled surface 18 may converge with the second beveled surface 20 to form a first cutting edge 24. In some embodiments, the second beveled surface 20 may converge with the third beveled surface 22 to form a second cutting edge 26. In some embodiments, the third beveled surface 22 may converge with the first beveled surface 18 to form a third cutting edge 28. In some embodiments, one or more of the first beveled surface 18, the second beveled surface 20, and the third beveled surface 22 may be symmetrical. In some embodiments, two of the first beveled surface 18, the second beveled surface 20, and the third beveled surface 22 may be inverted bevels.

As illustrated in FIG. 1E, in some embodiments, the first cutting edge 24 may be offset from the second cutting edge 26 by about 120 degrees. In some embodiments, the second cutting edge 26 may be offset from the third cutting edge 28 by about 120 degrees. In some embodiments, the third cutting edge 28 may be offset from the first cutting edge 24 by about 120 degrees. In some embodiments, the first cutting edge 24, the second cutting edge 26, and the third cutting edge 28 may trisection an outer circumference of the elongated body 14 into three equal sections, each of which may have a same shape or a different shape.

In some embodiments, a perimeter of the first beveled surface 18 may include the first cutting edge 24, the third cutting edge 28, and a first proximal edge 30. In some embodiments, the perimeter of the first beveled surface 18 may include no more than the first cutting edge 24, the third cutting edge 28, and the first proximal edge 30. In some embodiments, the first proximal edge 30 may extend between the first cutting edge 24 and the third cutting edge 28.

In some embodiments, a perimeter of the second beveled surface 20 may include the first cutting edge 24, the second cutting edge 26, and a second proximal edge 32. In some embodiments, the perimeter of the second beveled surface 20 may include no more than the first cutting edge 24, the second cutting edge 26, and the second proximal edge 32. In some embodiments, the second proximal edge 32 may extend between the second cutting edge 26 and the third cutting edge 28.

In some embodiments, a perimeter of the third beveled surface 22 may include the second cutting edge 26, the third cutting edge 28, and a third proximal edge 34. In some embodiments, the perimeter of the third beveled surface 22 may include no more than the second cutting edge 26, the third cutting edge 28, and a third proximal edge 34. In some embodiments, the third proximal edge 34 may extend between the second cutting edge 26 and the third cutting edge 28.

In some embodiments, the first cutting edge 24, the second cutting edge 26, and the third cutting edge 28 may be straight or linear. In some embodiments, the first cutting edge 24, the second cutting edge 26, and the third cutting edge 28 may intersect at the distal tip 16. In some embodiments, one or more of the following may be generally proximal edge-shaped: the first proximal edge 30, the second proximal edge 32, or the third proximal edge 34. In some embodiments, one or more of the following may include an arc: the first proximal edge 30, the second proximal edge 32, and the third proximal edge 34, which may facilitate smooth entry of the needle 10 through the skin and vein. In some embodiments, one or more of the following may be straight or linear: the first proximal edge 30, the second proximal edge 32, and the third proximal edge 34.

In some embodiments, one or more of the first perimeter, the second perimeter, and the third perimeter may be equal. In some embodiments, one or more of the first beveled surface 18, the second beveled surface 20, and the third beveled surface 22 may have equal shapes or contours, as illustrated, for example in FIGS. 1A-1E. In these and other embodiments, the distal tip 16 may be aligned with a central axis 36 of the needle 10. In some embodiments, one or more of the first beveled surface 18, the second beveled surface 20, and the third beveled surface 22 may be identical.

In some embodiments, a length 38 of the first beveled surface 18 from the distal tip to an apex of the first proximal edge 30 may be equal to a length 40 of the second beveled surface 20 from the distal tip 16 to an apex of the second proximal edge 32, which may be equal to a length 42 of the third beveled surface 22 from the distal tip 16 to an apex of the third proximal edge 34. In some embodiments, a length 44 of the first beveled surface 18 from a proximal end of the first cutting edge 24 to a proximal end of the third cutting edge 28 may be equal to a length 46 of the second beveled surface 20 from a proximal end of the first cutting edge 24 to a proximal end of the second cutting edge 26, which may be equal to a length 48 of the third beveled surface 22 from the proximal end of the second cutting edge 26 to the proximal end of the third cutting edge 28.

In some embodiments, a symmetrical nature of the distal end 12 may aid the user, who may be inexperienced in orientation of a needle for insertion into the patient. In some embodiments, the needle 10 may be solid. In some embodiments, the needle 10 may be constructed of metal, steel, or another suitable material. In some embodiments, the needle 10 may be monolithically formed as a single unit. In some embodiments, the distal tip 12 may not include an opening aligned with the central axis 36 of the needle 10. In some embodiments, the distal tip 12 may be closed and may not allow fluid to flow therethrough.

Referring now to FIGS. 1F-1G, in some embodiments, the length 38 of the first beveled surface 18, length 40 of the second beveled surface 20, and the length 46 of the second beveled surface 20 may be shorter compared to a length of a bevel of another needle 52, which is illustrated in dashed lines for size comparison. The other needle 52 may correspond to an ISO 10555 standard needle, which may include a reverse grind point geometry or a B-Bevel point geometry, as is known in the art. The other needle 52 may include an opening 53 along a central axis of the other needle 52.

In some embodiments, the length 38 of the first beveled surface 18, the length 40 of the second beveled surface 20, and the length 46 of the second beveled surface 20 may be about half or less of a length of the bevel of the other needle 52. In some embodiments, the length 38 of the first beveled surface 18, the length 40 of the second beveled surface 20, and the length 46 of the second beveled surface 20 may facilitate entry of a distal end 54 of the catheter 50 into the vein 49 before the distal tip 16 transfixes or damages an interior surface of a wall of the vein 49 opposite a point of entry of the needle 10 into the vein 49. In some embodiments, because the length 38 of the first beveled surface 18, the length 40 of the second beveled surface 20, and the length 46 of the second beveled surface 20 may be about half of the length of the bevel of the other needle 52, the distal tip 16 may be twice as far away from the interior surface of the wall of the vein 49 opposite the point of entry when the distal end 54 of the catheter 50 penetrates the vein 49.

In some embodiments, the distal end 12 of the needle 10 may be configured for difficult venous access, where a diameter of the vein is small, without damaging or transfixing the vein. In some embodiments, a distance 55 between an uppermost surface of the distal end 54 of the catheter 50 and a lowermost surface of the distal end 12 of the needle 10 may be half as much as a distance between an uppermost surface of the distal end 54 of the catheter 50 and a lowermost surface of a distal end of the other needle 52. In some embodiments, a distance 55 between an uppermost surface of the distal end 54 of the catheter 50 and a lowermost surface of the distal end 12 of the needle 10 may be about 1.1 mm or between 1.0 and 1.2 mm when the needle 10 is 20 gauge and configured for use with an 18 gauge catheter inserted at an insertion angle of 20 to 30 degrees. In some embodiments, a distance 55 between an uppermost surface of the distal end 54 of the catheter 50 and a lowermost surface of the distal end 12 of the needle 10 may be within three (3) standard deviations of a normal distribution of human vein diameters.

In contrast, the bevel of the other needle 48 may be so long as to make it difficult for the user to insert the distal end 54 of the catheter 50 into the vein 49 without damaging or transfixing the vein 49. Transfixing may occur when a needle enters a top of the vein 49 and then punctures a bottom of the vein 49 too. Damage to the vein 49, including transfixing the vein 49, may accelerate clotting and thrombosis. In some embodiments, a needle tip geometry of the needle 10 as described may provide a reduced risk of transfixing a vein 49 of a patient when the needle 10 is inserted into the vein 49 of the patient and may provide more reliable placement of the catheter 50 within the vein 49.

In some embodiments, the bevel of the other needle 52 may cause the other needle 52 to drop or dive at a steeper insertion angle as the other needle 52 is inserted through the skin and into the vein 49. The user may try to compensate for the steeper insertion angle by guessing at how much to lower the insertion angle. In some embodiments, the distal end 12 of the needle 10 may be symmetrical, which may provide zero drop upon insertion of the needle 10 into the patient.

In some embodiments, one or more of the first beveled surface 18, the second beveled surface 20, and the third beveled surface 22 may be generally planar. In some embodiments, the needle 10 may be oriented within the catheter 50 such that the first beveled surface 18, the second beveled surface 20, or the third beveled surface 22 faces downwardly or towards an interior wall of the vein 49 opposite a point of entry of the needle 10 into the vein 49, which may reduce a risk of transfixing the vein 49. Further, in some embodiments, use of the needle 10 may result in a cut or slit in the skin and the vein 49 a same or similar size as that of the other needle 52.

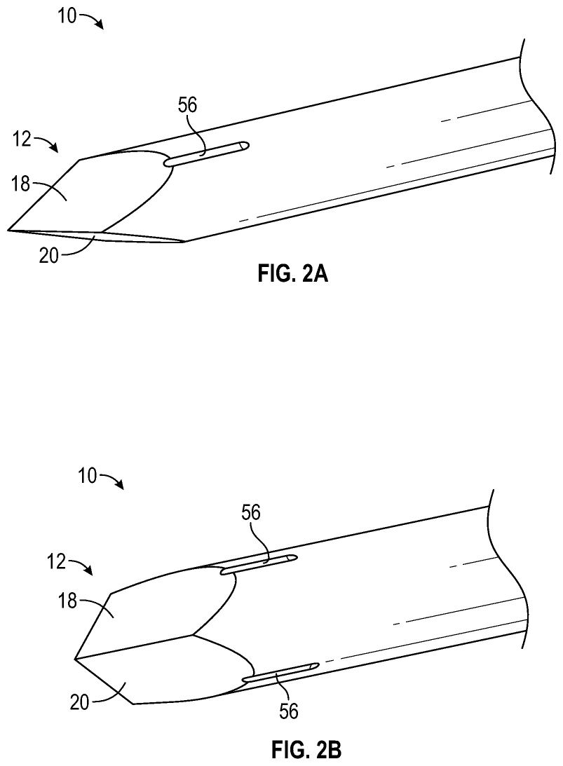

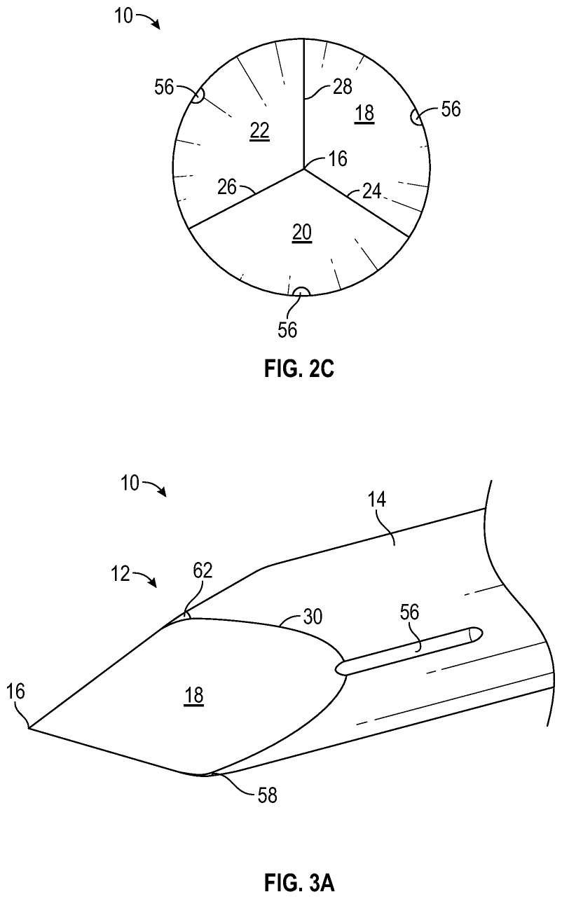

Referring now to FIGS. 2A-2C, in some embodiments, the needle 10 may include one or more flash grooves 56. In some embodiments, the flash grooves 56 may extend proximally from one or more of the first beveled surface 18, the second beveled surface 20, and the third beveled surface 22. In some embodiments, the flash grooves 56 may provide a blood flashback passage feature. In some embodiments, in response to the needle 10 penetrating the vein 49, blood may travel through the flash grooves 56 to an inner surface of the catheter 50 (illustrated, for example, in FIG. 1G) and/or to another location or device where the blood may be visualized by the user as an indication of placement of the needle 10 within the vein.

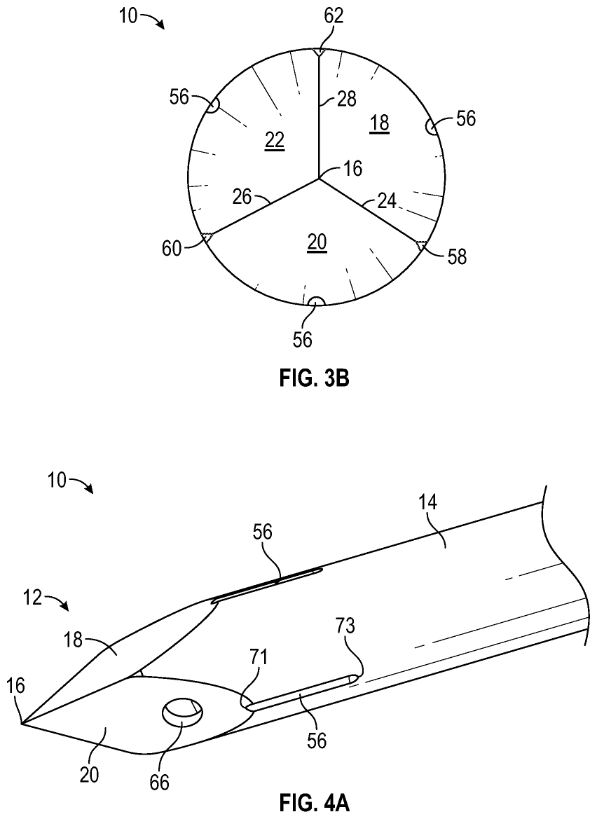

Referring now to FIGS. 3A-3B, in some embodiments, a first portion 58 of the elongated body 14 disposed between the first proximal edge 30 and the second proximal edge 32 may be chamfered. In some embodiments, a second portion 60 of the elongated body 14 between the second proximal edge 32 and the third proximal edge 34 may be chamfered. In some embodiments, a third portion 62 of the elongated body 14 disposed between the second proximal edge 32 and the third proximal edge 34 may be chamfered. In some embodiments, chamfering or rounding off of the first portion 58, the second portion 60, and the third portion 62 may prevent accidental slicing of the interior wall of the vein opposite a point of entry of the needle 10 into the vein.

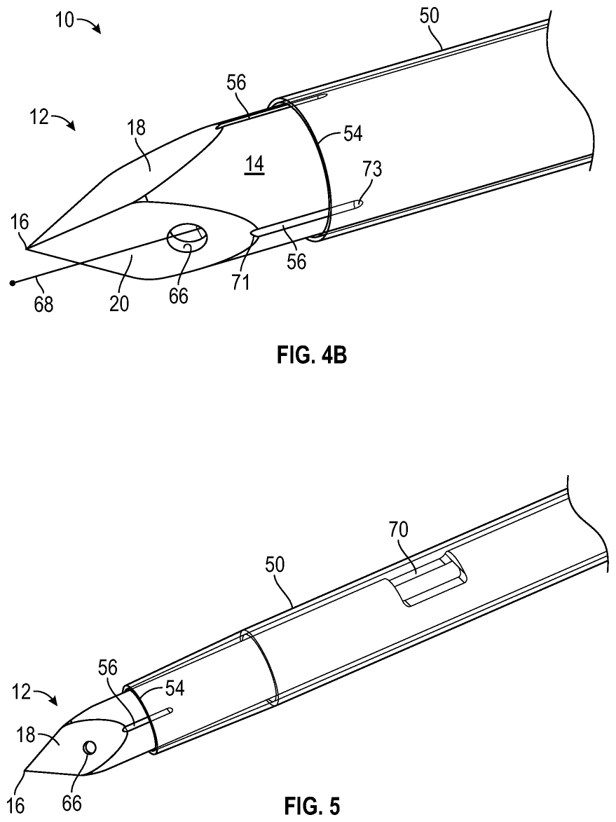

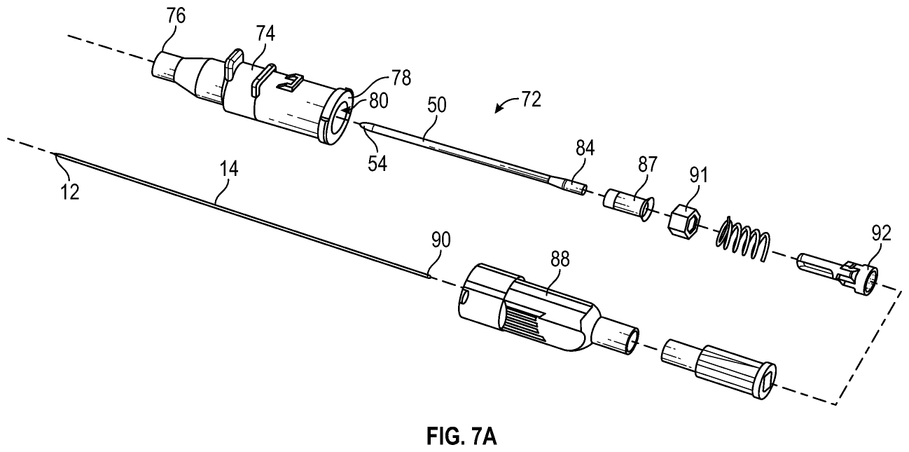

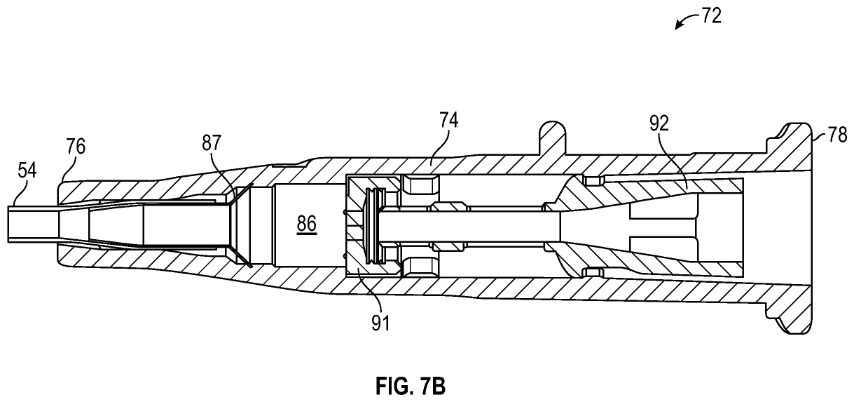

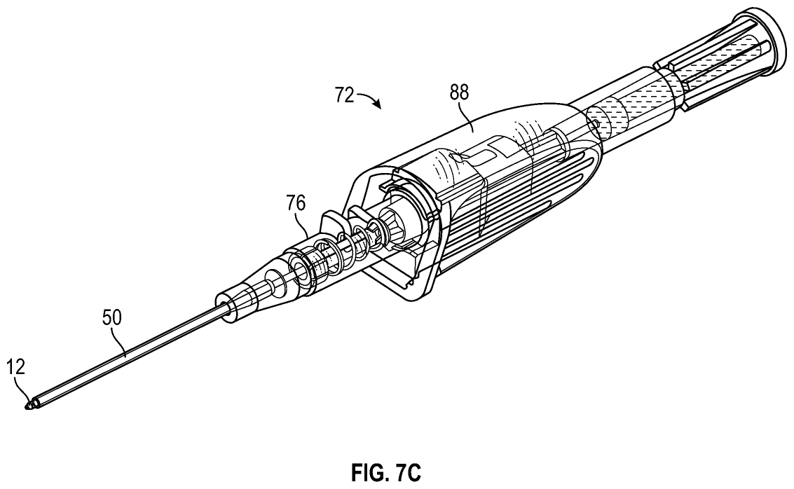

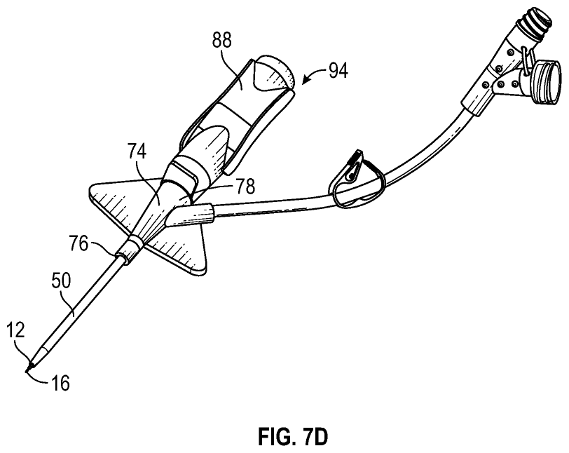

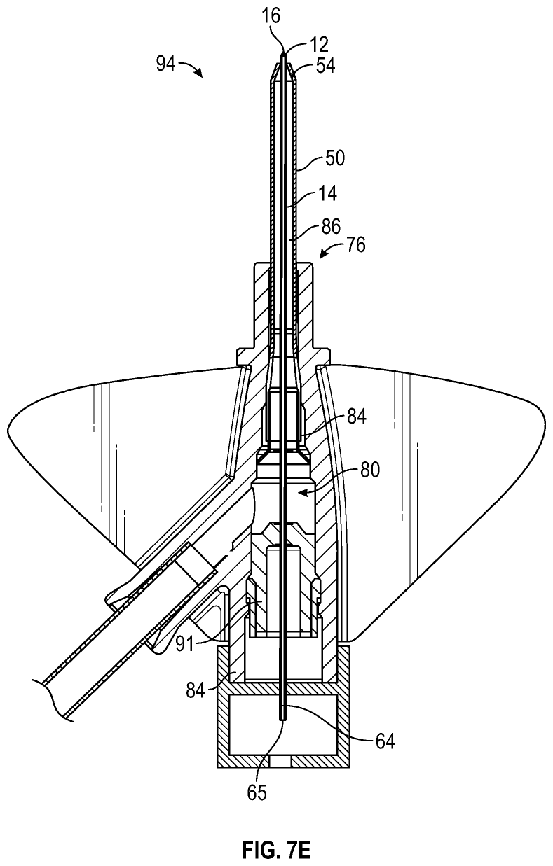

Referring now to FIGS. 4A-4B, in some embodiments, the needle 10 may include a lumen 64 extending through the proximal end 65 of the needle 10 (see, for example, FIG. 7E) via an opening, a notch, etc. In some embodiments, one or more of the first beveled surface 18, the second beveled surface 20, or the third beveled surface 22 may include one or more holes 66 in fluid communication with the lumen 64 of the needle 10. In some embodiments, a guidewire 68 may be disposed within the lumen 64 of the needle 10 and may be configured to extend through a particular one of the holes 66. In some embodiments, the guidewire 68 may be advanced distally through the particular one of the holes 66 beyond the distal tip 16 of the needle 10. In some embodiments, the needle 10 may be used to insert the guidewire 68 into the patient for advancing the catheter 50. In some embodiments, the distal tip 12 may be closed except for the holes 66 and may not allow fluid to flow therethrough except for the holes 66. In some embodiments, the holes 66 may be offset from the central axis 36.

Referring now to FIG. 5, in some embodiments, the needle 10 may include an opening, such as, for example, a notch 70, to provide flashback of blood when the needle 10 penetrates the vein to provide an indication that the distal tip 16 is positioned within the vein. In some embodiments, in response to the needle 10 penetrating the vein 49, blood may travel proximally through the holes 66, into the lumen 64 of the needle 10, and out the notch 70 to the inner surface of the catheter 50 and/or to another location or device where the blood may be visualized by the user as an indication of placement of the needle 10 within the vein.

In some embodiments, the flash grooves 56 may each include a distal end 71 proximate the first beveled surface 18, the second beveled surface 20, or the third beveled surface 22. In some embodiments, the distal end 71 may intersect a particular apex of a particular proximal edge. In some embodiments, the flash grooves 56 may each include a proximal end 73 disposed proximal to the distal end 54 of the catheter 50. In some embodiments, the flash grooves 56 may be in fluid communication with the lumen of the catheter 50. In some embodiments, blood traveling through the flash grooves 56 and into the lumen of the catheter 50 may be visible to the user.

As illustrated in FIG. 5, in some embodiments, the needle 10 may be oriented within the catheter 50 such that the first beveled surface 18, the second beveled surface 20, or the third beveled surface 22 faces downwardly or towards an interior wall of the vein 49 opposite a point of entry of the needle 10 into the vein 49, which may reduce a risk of transfixing the vein 49. In these embodiments, the notch 70 may be disposed facing a top of the catheter 50 and generally opposite the first beveled surface 18, the second beveled surface 20, or the third beveled surface 22, which may face downwardly.

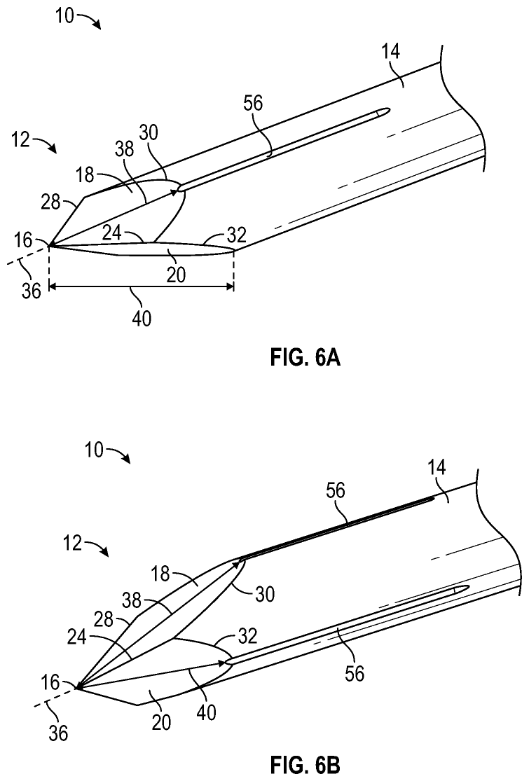

Referring now to FIGS. 6A-6B, in some embodiments, the distal end 12 of the needle 10 may be asymmetrical. In some embodiments, one or more of the first perimeter, the second perimeter, and the third perimeter may not be equal to each other. In these and other embodiments, the distal tip 16 may not be aligned with the central axis 36 of the needle 10.

In some embodiments, the length 38 of the first beveled surface 18 from the distal tip to the apex of the first proximal edge 30 may be greater than the length 40 of the second beveled surface 20 from the distal tip 16 to the apex of the second proximal edge 32 and/or the length 42 of the third beveled surface 22 from the distal tip 16 to the apex of the third proximal edge. In some embodiments, the length 40 of the second beveled surface 20 from the distal tip 16 to the apex of the second proximal edge 32 may be greater than the length 38 of the first beveled surface 18 from the distal tip 16 to the apex of the first proximal edge 30 and/or the length of the third beveled surface 22 from the distal tip 16 to the apex of the third proximal edge 34. In some embodiments, the length 42 of the third beveled surface 22 from the distal tip 16 to the apex of the third proximal edge 34 may be greater than the length 42 of the second beveled surface 20 from the distal tip 16 to the apex of the second proximal edge 32 and/or the length 38 of the first beveled surface 18 from the distal tip 16 to the apex of the first proximal edge 30.

As illustrated in FIG. 6A, in response to the length 38 of the first beveled surface 18 from the distal tip to the apex of the first proximal edge 30 being less than the length 40 of the second beveled surface 20 from the distal tip 16 to the apex of the second proximal edge 32 and/or the length 42 of the third beveled surface 22 from the distal tip 16 to the apex of the third proximal edge, the distal tip 16 may be offset from the central axis 36. As illustrated in FIG. 6B, in response to the length 38 of the first beveled surface 18 from the distal tip to the apex of the first proximal edge 30 being greater than the length 40 of the second beveled surface 20 from the distal tip 16 to the apex of the second proximal edge 32 and/or the length 42 of the third beveled surface 22 from the distal tip 16 to the apex of the third proximal edge, the distal tip 16 may be offset from the central axis 36.

Referring now to FIGS. 7A-7D, example blood flashback features in a catheter insertion device, including example catheter assembly 72, are illustrated, according to some embodiments. In some embodiments, the needle 10 may be used in the catheter assembly 72. It is understood that the needle 10 may be used with any catheter assembly, according to some embodiments.

In some embodiments, the catheter assembly 72 may include a straight or non-integrated catheter assembly. In some embodiments, the catheter assembly 72 may include an integrated catheter assembly. In further detail, in some embodiments, a catheter adapter 74 of the catheter assembly 72 may include an integrated extension tube, such as, for example, the BD NEXIVA™ Closed IV Catheter System, the BD NEXIVA™ DIFFUSICS™ Closed IV Catheter System, or the Becton Dickinson PEGASUS™ Safety Closed IV Catheter System.

In some embodiments, the catheter adapter 74 may include a distal end 76, a proximal end 78, a lumen 80 extending between the distal end 76 and the proximal end 78. In some embodiments, the catheter 50 may be secured to the catheter adapter 74 and may extend distally from the catheter adapter 74. In some embodiments, the catheter 50 may be held in place within the catheter adapter 74 via a wedge 87 or another suitable means. In some embodiments, the catheter 50 may include a PIVC, a midline catheter, or a PICC. In some embodiments, the catheter 50 may include the distal end 54, a proximal end 84, and a lumen 86 extending through the distal end 82 of the catheter 50 and the proximal end 84 of the catheter 50.

In some embodiments, the catheter assembly 72 may include a needle hub 88, in which the proximal end 65 of the needle 10 may be secured. In some embodiments, when the catheter assembly 72 is in an insertion position, ready for insertion into the patient, the needle 10 may extend through the catheter 50 such that the distal end 12 of the needle 10 is distal to the distal end 54 of the catheter 50. In some embodiments, after the catheter 50 is inserted into the vein of the patient, the needle 10 and the needle hub 88 may be removed from the catheter adapter 74, leaving the catheter 50 in the vein as the needle 10 is discarded. In some embodiments, a septum 91 and/or a septum actuator 92 may be disposed within the lumen 80 of the catheter adapter 74.

Flashback is the visibility of blood that confirms entry of the distal tip 16 into the vein. In some embodiments, flashback may be seen through the catheter 50 as blood travels through the flash grooves 56 (see, for example, FIGS. 2A-2C) and into a space between the needle 10 and the catheter 50. This flashback may be referred to as “Quickflash.” Additionally or alternatively, in some embodiments, flashback may be seen exiting the notch 70 (see, for example, FIG. 5) and entering the space between the needle 10 and the catheter 50. This may be referred to as “Instaflash.” Additionally or alternatively, in some embodiments, flashback may visible within a flash chamber, which may be disposed within the needle hub 88 or another suitable location within the catheter assembly 72.

In some embodiments, the needle 10 may be used with non-catheter medical devices and/or for any medical purpose, including as a spinal needle. In some embodiments, the needle 10 can also be used for other procedures, such as procedures where it is desired to reduce needle bending caused by a long bevel needle. In some embodiments, the needle 10 may be used for introducing and/or withdrawing fluid from a patient. In some embodiments, the needle 10 may be used for aspiration of blood, spinal fluid, or other fluids from a patient.

Referring now to FIGS. 7D-7E, another catheter assembly 94 with which the needle 10 may be used is illustrated, according to some embodiments. In some embodiments, the catheter assembly 94 may include or correspond to the catheter assembly 72. As illustrated in FIG. 7E, in some embodiments, the flash chamber may be disposed within the needle hub 88, which may be vented. In some embodiments, the needle 10 may be solid. In other embodiments, the holes 66 (see, for example, FIGS. 4A-4B) may provide access to the lumen 64 of the needle 10.

In some embodiments, the needle 10 may be used in coordination with various technologies. For example, in some embodiments, the distal tip 12 may be magnetic or magnetized and may allow ultrasound guidance and interaction with an ultrasound system to track a location of the distal tip 12.

In some embodiments, the needle 10 may include one or more features further described in U.S. patent application Ser. No. 15/286,223, filed Oct. 5, 2016, entitled “COMPLIANT CATHETER ADAPTER HAVING SELF-SLITTING NEEDLE,” which is hereby incorporated by reference in its entirety. In some embodiments, the needle 10 may include one or more features further described in U.S. patent application Ser. No. 15/286,162, filed Oct. 5, 2016, entitled “INTEGRATED CATHETER WITH INDEPENDENT FLUID PATHS,” which is hereby incorporated by reference in its entirety.

In some embodiments, the needle 10 may be used with a catheter having an asymmetric tip, as further described, for example, in U.S. patent application Ser. No. 15/286,261, filed Oct. 5, 2016, entitled “CATHETER WITH AN ASYMMETRIC TIP,” which is hereby incorporated by reference in its entirety. In some embodiments, the needle 10 may be used with a strain relief feature, which may be described, for example, in U.S. patent application Ser. No. 15/285,601, filed Oct. 5, 2016, entitled “CATHETER ADAPTER WITH DISTAL INNER DIAMETER CURVATURE PROVIDING KINK RESISTANCE,” which is hereby incorporated by reference in its entirety.

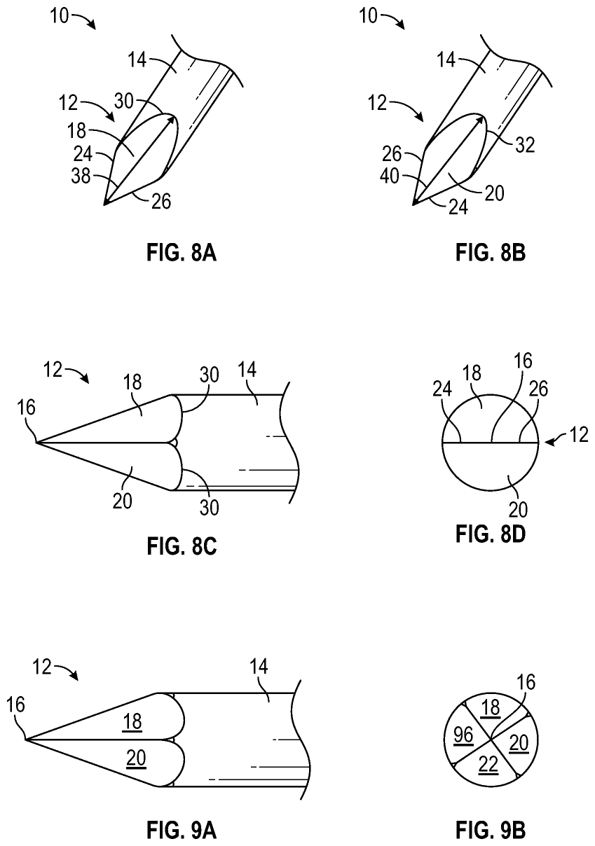

Referring now to FIGS. 8A-8D, in some embodiments, a number of beveled surfaces may vary. For example, as illustrated in FIGS. 8A-8D, the needle 10 may include two beveled surfaces, the first beveled surface 18 and the second beveled surface 20. In these and other embodiments, the needle 10 may include two cutting edges, the first cutting edge 24 and the second cutting edge 26, which may be offset from each other by about 180 degrees. As stated previously, in some embodiments, the first beveled surface 18 and the second beveled surface 20 may have equal shapes or contours. In some embodiments, the distal end 12 may be symmetric. In other embodiments, the distal end 12 may be asymmetrical. For example, the length 38 of the first beveled surface 18 from the distal tip to the apex of the first proximal edge 30 may be greater than the length 40 of the second beveled surface 20 from the distal tip 16 to the apex of the second proximal edge 32, as described. In some embodiments, the first beveled surface 18 and/or the second beveled surface 20 may include a particular hole 66.

Referring now to FIGS. 9A-9B, in some embodiments, the needle 10 may include more than three beveled surfaces. For example, as illustrated in FIGS. 9A-9B, the needle 10 may include four beveled surfaces and four cutting edges which may converge at the distal tip 16. FIG. 9B illustrates a fourth beveled surface 96, according to some embodiments. In some embodiments, the four beveled surfaces may be offset from each other by about 90 degrees. As another example, the needle 10 may include five beveled surfaces and five cutting edges which may converge at the distal tip 16. In some embodiments, the five beveled surfaces may be offset from each other by about 72 degrees. As yet another example, the needle 10 may include six beveled surfaces and six cutting edges which may converge at the distal tip 16. In some embodiments, the six beveled surfaces may be offset from each other by about 60 degrees.

In some embodiments, the four beveled surfaces, the five beveled surfaces, or the six beveled surfaces may have equal shapes or contours. In some embodiments, the distal end 12 may be symmetric. In other embodiments, the distal end 12 may be asymmetrical, such as, for example, with a beveled surface of the four beveled surfaces, the five beveled surfaces, or the six beveled surfaces being longer than another beveled surface of the four beveled surfaces, the five beveled surfaces, or the six beveled surfaces. In some embodiments, one or more particular beveled surface of the four beveled surfaces, the five beveled surfaces, or the six beveled surfaces may include the holes 66.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A catheter insertion device, comprising:
a needle, comprising a distal end, a proximal end, and an elongated body extending between the distal end and the proximal end, wherein the distal end comprises:
a distal tip;
a first beveled surface extending proximally from the distal tip;
a second beveled surface extending proximally from the distal tip; and
a third beveled surface extending proximally from the distal tip,
wherein the first beveled surface converges with the second beveled surface to form a first cutting edge, wherein the second beveled surface converges with the third beveled surface to form a second cutting edge, wherein the third beveled surface converges with the first beveled surface to form a third cutting edge,
wherein the first cutting edge is offset from the second cutting edge by about 120 degrees, wherein the second cutting edge is offset from the third cutting edge by about 120 degrees, wherein the third cutting edge is offset from the first cutting edge by about 120 degrees; and
a catheter, comprising a distal end, a proximal end, and a lumen extending through the distal end of the catheter and the proximal end of the catheter, wherein the needle extends through the catheter.

2. The catheter insertion device of claim 2, wherein the needle further comprises a flash groove, wherein the flash groove comprises a distal end proximate the first beveled surface, the second beveled surface, or the third beveled surface, wherein the flash groove further comprises a proximal end disposed proximal to the distal end of the catheter.

3. The catheter insertion device of claim 1, wherein the needle comprises a lumen extending through the proximal end of the needle, wherein the first beveled surface, the second beveled surface, or the third beveled surface comprises a hole in fluid communication with the lumen of the needle.

4. The catheter insertion device of claim 1, wherein a perimeter of the first beveled surface includes the first cutting edge, the third cutting edge, and a first proximal edge extending between the first cutting edge and the third cutting edge, wherein a perimeter of the second beveled surface includes the first cutting edge, the second cutting edge, and a second proximal edge extending between the second cutting edge and the third cutting edge, and wherein a perimeter of the third beveled surface includes the second cutting edge, the third cutting edge, and a third proximal edge extending between the second cutting edge and the third cutting edge.

5. The catheter insertion device of claim 4, wherein a first portion of the elongated body disposed between the first proximal edge and the second proximal edge is chamfered, a second portion of the elongated body between the second proximal edge and the third proximal edge is chamfered, or a third portion of the elongated body disposed between the second proximal edge and the third proximal edge is chamfered.

6. The catheter insertion device of claim 4, wherein a length of the first beveled surface from the distal tip to an apex of the first proximal edge is equal to a length of the second beveled surface from the distal tip to an apex of the second proximal edge is equal is a length of the third beveled surface from the distal tip to an apex of the third proximal edge.

7. The catheter insertion device of claim 4, wherein the perimeter of the first beveled surface, the perimeter of the second beveled surface, and the perimeter of the third beveled surface are equal.

8. The catheter insertion device of claim 4, wherein a length of the first beveled surface from the distal tip to an apex of the first proximal edge is greater than a length of the second beveled surface from the distal tip to an apex of the second proximal edge.

9. The catheter insertion device of claim 1, wherein the first beveled surface, the second beveled surface, and the third beveled surface are each planar.

10. The catheter insertion device of claim 1, wherein the needle is oriented within the catheter such that the first beveled surface, the second beveled surface, or the third beveled surface faces downwardly.

11. A needle, comprising:
a distal end, comprising:
a distal tip;
a first beveled surface extending proximally from the distal tip;
a second beveled surface extending proximally from the distal tip; and
a third beveled surface extending proximally from the distal tip, wherein the first beveled surface converges with the second beveled surface to form a first cutting edge, wherein the second beveled surface converges with the third beveled surface to form a second cutting edge, wherein the third beveled surface converges with the first beveled surface to form a third cutting edge, wherein the first cutting edge is offset from the second cutting edge by about 120 degrees, wherein the second cutting edge is offset from the third cutting edge by about 120 degrees, wherein the third cutting edge is offset from the first cutting edge by about 120 degrees;
a proximal end; and
an elongated body extending between the distal end and the proximal end.

12. The needle of claim 11, wherein the needle is solid.

13. The needle of claim 12, wherein the needle further comprises a flash groove extending proximally from the first beveled surface, the second beveled surface, or the third beveled surface.

14. The needle of claim 11, wherein the needle comprises a lumen extending through the proximal end of the needle, wherein the first beveled surface, the second beveled surface, or the third beveled surface comprises a hole in fluid communication with the lumen of the needle.

15. The needle of claim 11, wherein a perimeter of the first beveled surface includes the first cutting edge, the third cutting edge, and a first proximal edge extending between the first cutting edge and the third cutting edge, wherein a perimeter of the second beveled surface includes the first cutting edge, the second cutting edge, and a second proximal edge extending between the second cutting edge and the third cutting edge, and wherein a perimeter of the third beveled surface includes the second cutting edge, the third cutting edge, and a third proximal edge extending between the second cutting edge and the third cutting edge.

16. The needle of claim 15, wherein the perimeter of the first beveled surface, the perimeter of the second beveled surface, and the perimeter of the third beveled surface are equal, and the distal tip is aligned with a central axis of the needle.

17. The needle of claim 15, wherein the perimeter of the first beveled surface, the perimeter of the second beveled surface, and the perimeter of the third beveled surface are not equal, and the distal tip is not aligned with a central axis of the needle.

18. A needle, comprising:
a distal end, comprising:
a distal tip;
a first beveled surface extending proximally from the distal tip; and
a second beveled surface extending proximally from the distal tip,
wherein the distal end is closed along a central axis of the needle to prevent fluid from entering the distal end along the central axis.

19. The needle of claim 18, wherein the needle is solid.

20. The needle of claim 18, wherein the needle comprises a lumen extending through the proximal end of the needle, wherein the first beveled surface or the second beveled surface, comprises a hole offset from the central axis of the needle and in fluid communication with the lumen of the needle.
